(19) United States
(12) Patent Application Publication
(10) Pub. No.: US 2025/0275906 A1
(43) Pub. Date: Sep. 4, 2025

(54) METHOD FOR PREPARING LYOPHILIZED COLLAGEN POWDER AND USE THEREOF

(71) Applicant: Hangzhou Singclean Medical Products Co., Ltd, Hangzhou (CN)

(72) Inventors: Yun Huo, Hangzhou (CN); Xiaoqing Sun, Hangzhou (CN); Ningyong Si, Hangzhou (CN); Minmin Wu, Hangzhou (CN); Qiulin He, Hangzhou (CN); Wei Huang, Hangzhou (CN)

(21) Appl. No.: 18/650,404

(22) Filed: Apr. 30, 2024

(30) Foreign Application Priority Data
Feb. 29, 2024 (CN) 202410228306.6

(51) Int. Cl. A61K 8/65; A61L 2/00; A61L 24/10; A61Q 19/08

(52) U.S. Cl. CPC A61K 8/65; A61L 2/007; A61L 24/102; A61Q 19/08; A61K 2800/652; A61K 2800/84; A61L 2202/21; A61L 2400/04

(57) ABSTRACT

The present invention discloses a method for preparing a lyophilized collagen powder and use thereof. The preparation method includes the following steps: filling a certain amount of a collagen solution in an ice cube mold, and freezing in an ultra-low-temperature environment; crushing the frozen solid collagen in a low-temperature crusher, and performing quick lyophilization, to obtain an intermediate lyophilized collagen powder; further weighing an amount of a collagen solution, and uniformly mixing with the intermediate lyophilized collagen powder prepared in the previous step; repeating the low-temperature crushing operation with the mixed collagen solution, to obtain a lyophilized collagen powder; and obtaining a final product after filling and sterilization by freezing irradiation at a low temperature. The collagen powder product is useful for hemostasis, or tissue filling after dissolution in sterile physiological saline.

### CROSS-REFERENCE TO RELATED APPLICATION

The present application claims priority from Chinese Patent Application No. 202410228306.6 filed on Feb. 29, 2024, the contents of which are incorporated herein by reference in their entirety.

### TECHNICAL FIELD

The present invention relates to the field of medical devices and the field of medical beauty, and particularly to a lyophilized collagen powder, and a preparation method and use thereof.

### BACKGROUND

Collagen widely exists in animal skin, bones, tendons and other connective tissues, has high biological activity and function, and can participate in the cell migration, differentiation and proliferation, to allow the bones, tendons, cartilage and skin to have certain mechanical strength. Collagen is a fibrous protein with a unique triple helical structure. The unique structural features of collagen impart it with many useful performances, such as high tensile strength, controllable biodegradation, hemostasis, low antigenicity, low pro-inflammation and low cytotoxicity, and can promote the cell growth and adhesion, which make it an excellent natural biomedical material.

Hemostatic use: Collagen has a unique effect in promoting tissue regeneration and functional recovery, which is very beneficial to the healing and recovery of wounds after local hemostasis. Three types of products are commercially available now, including: sponge, powders, and meshes. It can be used for stop bleeding in all surgical departments. Various types of collagen are widely distributed in all the tissues of the body, but the distribution and content ratio of various types of collagen vary greatly from tissue to tissue. Therefore, it is very important to select the tissues rich in the required types of collagen molecules purposefully for obtaining sufficient collagen products with high purity. For collagen hemostatic powder products, the preparation method is relatively simple. As described in Chinese Invention Patent No. CN107349457A entitled “Preparation method of hemostatic collagen fibers”, a collagen solution is dehydrated over gradients, crushed, dried and crosslinked. Therefore, to meet the increasing demand for hemostatic collagen materials on the market, it is particularly important to propose a method for preparing a hemostatic collagen powder with simple operation and high recovery rate.

Use in medical beauty: Wrinkles, depressions, and scars, etc. in human facial skin can be treated by filling and remedy, to achieve the purpose of beautification. At present, hyaluronic acid has a large market share in the injection filling market. Compared with hyaluronic acid, collagen is different in molecular structure, performance characteristics and functions.

At present, the main components of collagen filler available on the market are collagen type I or collagen type III (mainly extracted from animal sources) which is pre-filled in a glass syringe in a suspension of 0.3% lidocaine hydrochloride in physiological saline, and stored in a freezer. In addition, two lyophilized recombinant collagen powders or solution fillers are available.

Currently, collagen fillers generally have the following technical difficulties:
(1) Commercially available collagen fillers pre-filled in glass syringes are all sterilized by filtration, and are not suitable for terminal sterilization by, for example, irradiation sterilization or moist-heat sterilization, so safer and more reliable sterilization cannot be guaranteed. (2) Collagen is susceptible to bacterial contamination in a solution environment, so cold chain transportation and cryopreservation are needed, and the cost is thus high. The conventional collagen solution cannot be used to prepare collagen stored at room temperature by a lyophilization process, because for active collagen with high molecular weight, if it is simply lyophilized, collagen will undergo irreversible self-crosslinking during the lyophilization process, resulting in agglomerated collagen, which is difficult to dissolve in physiological saline and other equilibrium solutions. (3) Collagen can be obtained by acid dissolution and acid dissolution-enzymatic method, and can only be re-dissolved in an acid solution over a long period of time. Moreover, collagen in the product is dispersed in a balanced salt solution, which needs to be mixed uniformly and pre-filled in a glass syringe in advance.

### SUMMARY

To solve the problems existing in the prior art, an object of the present invention is to provide a method for preparing a lyophilized collagen powder, a prepared lyophilized collagen powder, and use thereof.

According to a first aspect of the present invention, the following technical solutions are adopted in the present invention.

A method for preparing a lyophilized collagen powder is provided, which includes the following steps:
(1) weighing a certain amount of a collagen solution, filling in an ice cube mold, and freezing in an ultra-low-temperature environment, to ensure that the collagen solution is always in a solid state before entering Step (2), where further, the cryopreservation temperature is −80° C. to −20° C., and the cryopreservation time is 10-20 hrs; preferably, the cryopreservation temperature is −40° C. to −20° C.; further preferably, the cryopreservation temperature is −30° C.; and the cryopreservation time is preferably 15 hrs;
(2) crushing the frozen solid collagen in a low-temperature crusher, at a temperature that ensures the solid collagen to still remain solid during the crushing, where further, the temperature during low-temperature crushing is −30° C. to −5° C., and preferably, the temperature during low-temperature crushing is −10° C.;
(3) quickly lyophilizing the crushed solid collagen, and removing the crushed solid collagen after reaching room temperature, to obtain an intermediate lyophilized collagen powder, where further, during the quick lyophilization, the pre-freezing temperature is −20° C. to −80° C., the pre-freezing time is 4 to 12 hrs, and the lyophilizing time is 48 to 96 hrs; and preferably, the pre-freezing temperature is −30° C.; the pre-freezing time is 6 hrs, and the lyophilizing time is 72 hrs;
(4) further weighing a certain amount of a collagen solution, uniformly mixing the obtained intermediate lyophilized collagen powder in the collagen solution, and stirring at a low temperature, to mix them uniformly, where further, the weight ratio of the solid contents in the collagen solutions in Step (1) and Step (4) is 1:0.5-2:1, the low-temperature stirring temperature is preferably 2° C.-10° C., and the low-temperature stirring time is preferably 12 to 24 hrs; and further preferably, the weight ratio of the solid contents in the collagen solutions in Step (1) and Step (4) is 1:1, and the low-temperature stirring temperature is 4° C.
(5) filling the mixed collagen solution in an ice cube mold, and repeating the processes from Step (1) to Step (3), to obtain an intermediate lyophilized collagen powder with increased solid content; and
(6) taking the intermediate lyophilized collagen powder with increased solid content as a lyophilized collagen powder where the solid content meets the required standard, and subjecting the lyophilized collagen powder to terminal sterilization to obtain a final lyophilized collagen powder product.

Further, the terminal sterilization is irradiation sterilization after the lyophilized collagen powder is filled. Further, freezing irradiation at a low temperature in the presence of dry ice is adopted, where the dose of irradiation sterilization is 10 to 25 KGy, and the temperature is −10° C. to −70° C.

According to a second aspect of the present invention, the present invention provides a lyophilized collagen powder prepared by any one of the above preparation methods.

According to a third aspect of the present invention, the present invention provides use of a lyophilized collagen powder prepared by any one of the above preparation methods, in a homeostatic powder in surgery and as a tissue filler.

When used for hemostasis in surgery and tissue filling in medical beauty, the lyophilized collagen powder after irradiation at a low temperature is packaged and stored. When used as a filler, it can be dissolved in physiological saline, water for injection or a buffer, and used for tissue filling of the face, nose and lips.

Therefore, in view of the problems existing in the prior art, the present invention provides a lyophilized collagen powder with excellent performance in urgent need in the market, which (1) is sterilized by terminal sterilization, to achieve a high sterility level; (2) is stored at normal temperature; (3) is a high-molecular-weight collagen, having a longer maintenance effect; (4) retains the structure of natural collagen without denaturization, to ensure the safety and effectiveness during use; (5) has simple composition and low solvent residue; (6) has good hemostatic performance; and (7) can be quickly dispersed when used as a filler.

The method steps adopted in the present invention are designed based on physical processing technologies, thus avoiding the risks caused by chemical reagents and solvent residues. By controlling the temperature of the whole dehydration and powder forming process not to exceed 10° C., collagen denaturation is avoided in the present invention, to greatly ensure the purity of collagen extracted and reduce the loss of raw materials. In the present invention, terminal irradiation sterilization is employed, so that the product reaches a sterility level of 10⁻⁶. The product of the present invention can be stored at normal temperature, thus reducing the energy consumption caused by storage and transportation. The product of the present invention can be quickly dispersed when used in medical beauty, thus shortening the operation and waiting time of a medical staff.

### BRIEF DESCRIPTION OF THE DRAWINGS

FIG. 1 is an SDS polyacrylamide gel electrophoresis diagram of a lyophilized collagen powder in Example 1 of the present invention.

FIG. 2(a) is a physical picture of the lyophilized collagen powder in Example 1 of the present invention.

FIG. 2(b) is a physical picture of the lyophilized collagen powder in Example 1 of the present invention reconstituted in sterile physiological saline.

FIG. 3 is a physical picture of a syringe filled with the lyophilized collagen powder in Example 1 of the present invention reconstituted in sterile physiological saline.

### DETAILED DESCRIPTION

To facilitate the further understanding of the present invention, the lyophilized collagen powder provided in the present invention will be further described below by way of examples. However, the present invention is not limited thereto. Non-essential improvements and adjustments made by technicians in the art under the guidance of the core idea of the present invention still fall within the scope of protection of the present invention.

### (I) Method for Preparing a Lyophilized Collagen Powder

### Example 1

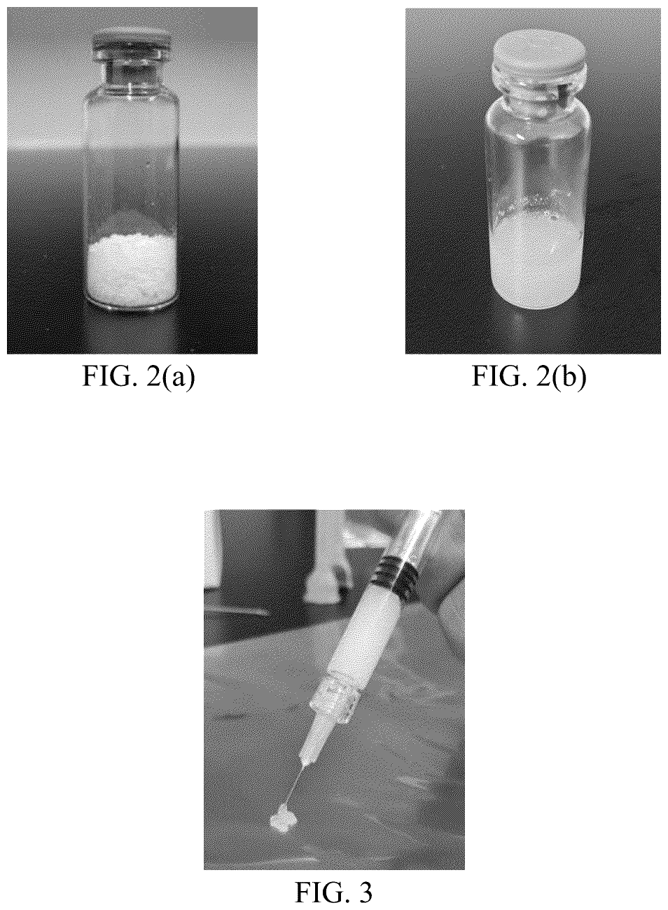

(1) 300 g of a collagen solution was weighed, and filled in an ice cube mold by using a syringe, and then the ice cube mold was allowed to stand in an ultra-low-temperature freezer at −30° C. for 15 hrs. The collagen solution was a stock collagen solution, and the potential allergic risk was completely removed by virus inactivation treatment and enzyme digestion to remove the terminal peptide.
(2) A low-temperature constant-temperature reaction bath was started, and the temperature was set to make the actual temperature of the reaction bath be −10° C. A low-temperature crusher was connected, and the frozen collagen solid was added into the crusher and crushed into a white snowflake shape;
(3) The crushed sample was transferred to an ultra-low-temperature freezer, pre-frozen at −30° C. for 6 hrs, and then dried in a freeze dryer for 72 hrs, to obtain an intermediate lyophilized collagen powder.
(4) The low-temperature constant-temperature reaction bath was started again, and the temperature was set to make the actual temperature of the reaction bath be 4° C. 300 g of a collagen solution was weighed, and the solid powder obtained in Step (3) was uniformly mixed in the collagen solution, and stirred for 12 hrs at a low temperature, to mix them uniformly. The weight ratio of the solid contents of the collagen solutions in Step (1) and Step (4) was 1:2.
(5) The reconstituted solution was filled in an ice cube mold, and then the ice cube mold was allowed to stand in an ultra-low-temperature freezer at −30° C. for 15 hrs. The processes from Step (2) to Step (3) were repeated, to obtain a lyophilized collagen powder. The lyophilized collagen powder was filled, and underwent freezing irradiation at a low temperature in the presence of dry ice, where the irradiation dose was 15 KGy, and the temperature was-40° C. Finally, a final lyophilized collagen powder product was obtained, as shown in FIG. 2(a).
(6) 20 mg of the lyophilized collagen powder obtained in Step (5) was dissolved in 2 ml of sterile physiological saline, and vortexed for 1 min to obtain a reconstituted material as shown in FIG. 2(b).
(7) 1 ml of the reconstituted material obtained in Step (6) was extracted with a 1.25 ml sterile syringe, and then and then pushed from the syringe through a 27G needle, for simulated injection, as shown in FIG. 3.

### Example 2

(1) 425 g of a collagen solution was weighed, and filled in an ice cube mold by using a syringe, and then the ice cube mold was allowed to stand in an ultra-low-temperature freezer at −80° C. for 10 hrs. The collagen solution was a stock collagen solution, and the potential allergic risk was completely removed by virus inactivation treatment and enzyme digestion to remove the terminal peptide.
(2) A low-temperature constant-temperature reaction bath was started, and the temperature was set to make the actual temperature of the reaction bath be −15° C. A low-temperature crusher was connected, and the frozen collagen solid was added into the crusher and crushed into a white snowflake shape.
(3) The crushed sample was transferred to an ultra-low-temperature freezer, pre-frozen at −30° C. for 6 hrs, and then dried in a freeze dryer for 72 hrs, to obtain an intermediate lyophilized collagen powder.
(4) The low-temperature constant-temperature reaction bath was started again, and the temperature was set to make the actual temperature of the reaction bath be 4° C. 300 g of a collagen solution was weighed, and the solid powder obtained in Step (3) was uniformly mixed in the collagen solution, and stirred for 12 hrs at a low temperature, to mix them uniformly. The weight ratio of the solid contents of the collagen solutions in Step (1) and Step (4) was 1:1.5.
(5) The reconstituted solution was filled in an ice cube mold, and then the ice cube mold was allowed to stand in an ultra-low-temperature freezer at −80° C. for 10 hrs. The processes from Step (2) to Step (3) were repeated, to obtain a lyophilized collagen powder. The lyophilized collagen powder was filled, and underwent freezing irradiation at a low temperature in the presence of dry ice, where the irradiation dose was 15 KGy, and the temperature was-30° C. Finally, a final lyophilized collagen powder product was obtained.

### Example 3

(1) 360 g of a collagen solution was weighed, and filled in an ice cube mold by using a syringe, and then the ice cube mold was allowed to stand in an ultra-low-temperature freezer at −50° C. for 12 hrs. The collagen solution was a stock collagen solution, and the potential allergic risk was completely removed by virus inactivation treatment and enzyme digestion to remove the terminal peptide.
(2) A low-temperature constant-temperature reaction bath was started, and the temperature was set to make the actual temperature of the reaction bath be −10° C. A low-temperature crusher was connected, and the frozen collagen solid was added into the crusher and crushed into a white snowflake shape.
(3) The crushed sample was transferred to an ultra-low-temperature freezer, pre-frozen at −20° C. for 8 hrs, and then dried in a freeze dryer for 72 hrs, to obtain an intermediate lyophilized collagen powder.
(4) The low-temperature constant-temperature reaction bath was started again, and the temperature was set to make the actual temperature of the reaction bath be 6° C. 450 g of a collagen solution was weighed, and the solid powder obtained in Step (3) was uniformly mixed in the collagen solution, and stirred for 24 hrs at a low temperature, to mix them uniformly. The weight ratio of the solid contents of the collagen solutions in Step (1) and Step (4) was 1:2.
(5) The reconstituted solution was filled in an ice cube mold, and then the ice cube mold was allowed to stand in an ultra-low-temperature freezer at −50° C. for 12 hrs. The processes from Step (2) to Step (3) were repeated, to obtain a lyophilized collagen powder. The lyophilized collagen powder is filled, and underwent freezing irradiation at a low temperature in the presence of dry ice, where the irradiation dose was 20 KGy, and the temperature was-40° C. Finally, a final lyophilized collagen powder product was obtained.

### Example 4

(1) 575 g of a collagen solution was weighed, and filled in an ice cube mold by using a syringe, and then the ice cube mold was allowed to stand in an ultra-low-temperature freezer at −40° C. for 13 hrs.
(2) A low-temperature constant-temperature reaction bath was started, and the temperature was set to make the actual temperature of the reaction bath be −15° C. A low-temperature crusher was connected, and the frozen collagen solid was added into the crusher and crushed into a white snowflake shape.
(3) The crushed sample was transferred to an ultra-low-temperature freezer, pre-frozen at −40° C. for 4 hrs, and then dried in a freeze dryer for 60 hrs, to obtain an intermediate lyophilized collagen powder.
(4) The low-temperature constant-temperature reaction bath was started again, and the temperature was set to make the actual temperature of the reaction bath be 2° C. 500 g of a collagen solution was weighed, and the solid powder obtained in Step (3) was uniformly mixed in the collagen solution, and stirred for 12 hrs at a low temperature, to mix them uniformly. The weight ratio of the solid contents of the collagen solutions in Step (1) and Step (4) was 1:1.7.
(5) The reconstituted solution was filled in an ice cube mold, and then the ice cube mold was allowed to stand in an ultra-low-temperature freezer at −40° C. for 13 hrs. The processes from Step (2) to Step (3) were repeated, to obtain a lyophilized collagen powder. The lyophilized collagen powder is filled, and underwent freezing irradiation at a low temperature in the presence of dry ice, where the irradiation dose was 25 KGy, and the temperature was-50° C. Finally, a final lyophilized collagen powder product was obtained.

### Comparative Example 1

In this comparative example, lyophilization was still used. However, Steps (1), (2) (3) (4) and (5) in Examples 1, 2, 3 and 4 were omitted. The preparation steps in Comparative Example 1 were as follows:
(D1) An amount of a collagen solution with the same concentration as that in the examples was weighed, and evenly spread on a stainless steel plate.
(D2) The stainless steel plate was dried for 72 hrs in a freeze dryer, to obtain a lyophilized collagen product.
(D3) The lyophilized product was cut, packaged with a plastic container, and subjected to irradiation sterilization to obtain a final lyophilized collagen sponge product.

### (II) Detection of Physical and Chemical Properties and Indexes of Final Lyophilized Collagen Powder Product

The detection method was as follows:

### (1) Residue on Ignition

The residue on ignition was determined according to Chinese Pharmacopoeia (2020 edition), Vol. IV, General rule 0841-Residue on ignition test method. Performance requirements: The sulfate ash content should be no more than 2.0% according to YY/T 0606.6-2008 Tissue Engineering Medical Products, Part 6, Type I Collagen, and the residue on ignition of an implant should be no more than 10 mg/g (mass fraction), that is, no more than 1.0%, according to YY/T 0954-2015 Non-active Surgical Implants-Type I Collagen Implants.

### (2) Protein Content

The protein content was determined according to Chinese Pharmacopoeia (2020 edition), Vol. IV, General rule 0731-Protein Content Determination method. Performance requirements: The collagen purity should be ≥95% (m/m) according to YY/T 0606.6-2008 Tissue Engineering Medical Products, Part 6, Type I Collagen, and the total amount of impurity proteins in the implant should be less than 1% of the total proteins according to YY/T 0954-2015 Non-active Surgical Implants-Type I Collagen Implants.

### (3) Hydroxyproline Content

The hydroxyproline content was determined according to YY/T 1453-2016 Tissue engineering medical device products. Methods for determination of type I collagen. Appendix B. Method I. Performance requirement: The hydroxyproline content should not be less than 9% (m/m) of the total protein content according to YY/T 0606.6-2008 Tissue Engineering Medical Products, Part 6, Type I Collagen.

### (4) Loss on Drying

The loss on drying was determined according to Chinese Pharmacopoeia (2020 edition), Vol IV, General rule 0831-Determination of Loss on Drying. Performance requirement: The loss on drying should be no more than 15.0% according to YY/T 1511-2017 Collagen Sponge.

(5) pH

The protein content was determined according to Chinese Pharmacopoeia (2020 edition), Vol. IV, General rule 0631-pH Determination method. Performance requirements: The pH should be greater than 4.0 according to YY/T 0606.6-2008 Tissue Engineering Medical Products, Part 6, Type I Collagen, and the pH of the implant should be in the range of 6.0-8.0, according to YY/T 0954-2015 Non-active Surgical Implants-Type I Collagen Implants.

### (6) Heavy Metal Content

The heavy metal content was determined according to Chinese Pharmacopoeia (2020 edition), Vol. IV, General rule 0821-Heavy metal test method. Performance requirements: The total heavy metal content (based on lead) should be not greater than 10 μg/g (mass fraction) according to YY/T 0606.6-2008 Tissue Engineering Medical Products, Part 6, Type I Collagen, and YY/T 0954-2015 Non-active Surgical Implants-Type I Collagen Implants.

### (7) Sterility Test

The sterility was test according to Chinese Pharmacopoeia (2020 edition), Vol. IV, General rule 1101-Sterility test method. Performance requirements: The product should be sterile according to YY/T 0606.6-2008 Tissue Engineering Medical Products, Part 6, Type I Collagen, and YY/T 0954-2015 Non-active Surgical Implants-Type I Collagen Implants.

### (8) Bacterial Endotoxin Content

The bacterial endotoxin content was determined according to Chinese Pharmacopoeia (2020 edition), Vol. IV, General rule 1143-Bacterial endotoxin test method. Performance requirement: The bacterial endotoxin content in the implant should be less than 0.5 EU/ml according to YY/T 0954-2015 Non-active Surgical Implants-Type I Collagen Implants.

### (9) Identification

The identification was performed according to Chinese Pharmacopoeia (2020 edition), Vol. IV, General rule 0541: SDS-polyacrylamide gel electrophoresis method.

The test results are as follows:

TABLE 1
Physical and chemical property test of final lyophilized collagen powder product

| Test item | Example 1 | Example 2 | Example 3 | Example 4 |
|---|---|---|---|---|
| Appearance | White loose | White loose | White loose | White loose |
| | lumps or | lumps or | lumps or | lumps or |
| | powder, | powder, | powder, | powder, |
| | containing no | containing no | containing no | containing no |
| | foreign matter | foreign matter | foreign matter | foreign matter |
| | visible to the | visible to the | visible to the | visible to the |
| | naked eyes | naked eyes | naked eyes | naked eyes |
| Residue on ignition | 0.6% | 0.66% | 0.1% | 0.2% |
| Protein content | 99.2% | 99.4% | 99.5% | 99% |
| Hydroxyproline | 14% | 12% | 12% | 12% |
| content | | | | |
| Loss on drying | 13% | 11.0% | 7.7% | 10.4% |
| pH | 6.31 | 6.35 | 6.28 | 6.26 |
| Heavy metal content | Accepted | Accepted | Accepted | Accepted |
| Sterility test | Sterility | Sterility | Sterility | Sterility |
| Bacterial endotoxin | <0.5 EU/ml | <0.5 EU/ml | <0.5 EU/ml | <0.5 EU/ml |
| content | | | | |

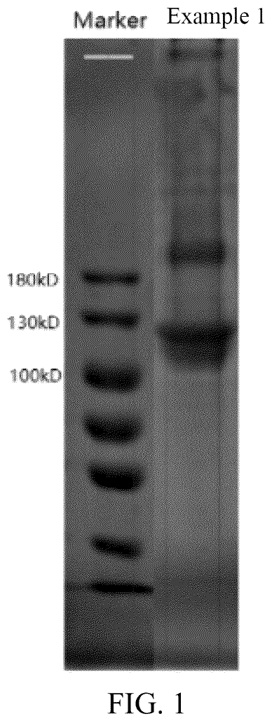

Identification: FIG. 1 is an SDS polyacrylamide gel electrophoresis diagram of a lyophilized collagen powder in Example 1 of the present invention, showing that the product retains the triple helical structure of collagen, and has a molecular weight of greater than 30 kD.

### (III) In-Vitro Cytotoxicity Test of Final Lyophilized Collagen Powder Product Prepared in Examples

Test method: The in-vitro biological safety test was carried out according to GBT 16886.5-2017 “Biological Evaluation of Medical Devices Part 5, Test for In-vitro Cytotoxicity”. Mouse fibroblasts were used. The negative control group was exchanged with a stainless steel material extract, the positive control group was exchanged with a 5% DMSO extract, the test group was exchanged with a sample extract of Example 1, and the blank control group was exchanged with fresh cell culture. The results show that the positive control group has severe cytotoxicity, the negative control group has no cytotoxicity, the test group has no significant difference compared with the negative control group, and has no cytotoxicity. The specific test results are shown in Table 2.

TABLE 2
In-vitro cytotoxicity test

| Test item | Test group | Positive control group | Negative control group |
|---|---|---|---|
| Cell state | The cells has normal | The cells are round up, | The cell morphology |
| | morphology, and grow | detached and found | is normal, and grow |
| | well | dead | well |
| Relative | 108.9% | 11.5% | 118.7% |
| proliferation rate | | | |

### (IV) In-Vitro Degradation Test of Final Lyophilized Collagen Powder Product Prepared in Examples

Test method: The in-vitro degradation test was carried out according to GB/T16886.13-2017 “Biological Evaluation of Medical Devices-Part 13: Identification and Quantification of Degradation Products from Polymeric Medical Products”. Before the test, the samples of the above examples were dried to a constant weight, and then 12 portions of each of the samples (with roughly the same weight) were weighed by an analytical balance, respectively added to a buffer, taken out after 2, 7, 30 and 90 days, dried to a constant weight and weighed. The weight loss of the samples before and after degradation was compared. Weight loss rate=(weight before degradation-weight after degradation)/weight before degradation×100%. The specific test results are shown in Table 3.

TABLE 3
In-vitro degradation test of lyophilized collagen powder

| Test item | Example 1 | Example 2 | Example 3 | Example 4 |
|---|---|---|---|---|
| Weight loss rate | 7.2% | 6.8% | 7.3% | 7.1% |
| after 2 days of in- | | | | |
| vitro degradation | | | | |
| Weight loss rate | 15.4% | 14.6% | 15.8% | 15.7% |
| after 7 days of in- | | | | |
| vitro degradation | | | | |
| Weight loss rate | 42.7% | 40.5% | 43.4% | 42.9% |
| after 30 days of in- | | | | |
| vitro degradation | | | | |
| Weight loss rate | 53.6% | 52.9% | 55.2% | 54.3% |
| after 90 days of in- | | | | |
| vitro degradation | | | | |

### (V) Hemostatic Performance Test of Lyophilized Collagen Powder Prepared in Examples and Comparative Example when Used in Hemostatic Powders

### (1) Liquid Absorbability

A 500-mesh screen was folded into a small rectangle or square, put into water for absorption for 1 min, and then taken out. The excess water on the outer surface was absorbed by absorbent paper, then the screen was weighed, and the weight was recorded as m₁. The sample was precisely weighed and the weight was recorded as m₂. Then the sample was put in water and taken out after fully absorption. The water on the surface was absorbed by absorbent paper, then the sample was weighed and the weight was recorded as m₃. Liquid absorbability=(m₃−m₁)/m₂. The specific test results are shown in Table 4.

### (2) Hemostasis Time

15 SD rats were randomly divided into 3 groups, weighed and anesthetized by injecting 3% pentobarbital sodium via the tail vein. The rats were cut along the midabdominal line by surgical scissors, with an opening of about 2.5 cm. The right external iliac artery was separated, and pierced to bleed by a sterile needle. The oozing blood was wiped off with a sterile cotton ball. The sample of Example 3, the sample of Comparative Example 1 and a model control sample (commercial sterile gauze) of similar weights were respectively attached to the bleeding site and timed until the bleeding was completely stopped. The specific test results are shown in Table 4.

TABLE 4
Hemostatic performance test

| | | | |
|---|---|---|---|
| | | Comparative | Model |
| Test item | Example 3 | Example 1 | control group |

| | | | | | | |
|---|---|---|---|---|---|---|
| Liquid | 161 | times | 65 | times | 30 | times |
| absorbability | | | | | | |
| Hemostasis | 30 | s | 70 | s | 100 | s |
| time | | | | | | |

### (VI) Solubility Test of Final Lyophilized Collagen Powder Products Prepared in Examples and Comparative Examples as Fillers

Test method: 90 mg of each of the samples of the examples and comparative examples and 3 ml of sterile physiological saline were weighed into a penicillin bottle, and fully dissolved by vortex. The time spent for complete dissolution and the state of the samples after standing were recorded. The test results are shown in Table 5.

TABLE 5
Solubility test of lyophilized collagen filler

| | | | | | Comparative |
|---|---|---|---|---|---|
| Test item | Example 1 | Example 2 | Example 3 | Example 4 | Example 1 |
| Time for | Complete | Complete | Complete | Complete | Failed to complete |
| complete | dissolution in | dissolution | dissolution | dissolution | dissolve |
| dissolution | 30 s | in 30 s | in 30 s | in 30 s | |
| State of the | Clear | Clear | Clear | Clear | Undissolved white |
| dissolved | solution | solution | solution | solution | lumpy solid is |
| samples after | | | | | present in the |
| standing | | | | | solution |

Specific embodiments of the present invention have been described above; however, the technical features of present invention are not limited thereto. Any change or modification made by a person skilled in the relevant art without departing from the spirit of the present invention is covered by the scope of protection of the present invention.

What is claimed is:

1. A method for preparing a lyophilized collagen powder, comprising the following steps:
(1) weighing a certain amount of a collagen solution, filling in an ice cube mold, and freezing in an ultra-low-temperature environment,
(2) crushing the frozen solid collagen in a low-temperature crusher, at a temperature that ensures the solid collagen to still remain solid during the crushing;
(3) quickly lyophilizing the crushed solid collagen, and removing the crushed solid collagen after reaching room temperature, to obtain an intermediate lyophilized collagen powder;
(4) further weighing a certain amount of a collagen solution, uniformly mixing the obtained intermediate lyophilized collagen powder in the collagen solution, and stirring at a low temperature, to mix them uniformly;
(5) filling the mixed collagen solution in an ice cube mold, and repeating the processes from Step (1) to Step (3), to obtain an intermediate lyophilized collagen powder with increased solid content; and
(6) taking the intermediate lyophilized collagen powder with increased solid content as a lyophilized collagen powder where the solid content meets the required standard, and subjecting the lyophilized collagen powder to terminal sterilization to obtain a final lyophilized collagen powder product.

2. The method for preparing a lyophilized collagen powder according to claim 1, wherein the terminal sterilization is irradiation sterilization after the lyophilized collagen powder is filled.

3. The method for preparing a lyophilized collagen powder according to claim 1, where if the lyophilized collagen powder with increased solid content obtained after Step (4) and Step (5) are performed once does not meet the solid content requirement, Step (4) and Step (5) are repeated, until the solid content meets a required standard.

4. The method for preparing a lyophilized collagen powder according to claim 1, wherein in step (1), the freezing temperature is −80° C. to −20° C., and the freezing time is 10-20 hrs;
preferably, the freezing temperature is −40° C. to −20° C.; further preferably, the freezing temperature is −30° C.; and the freezing time is preferably 15 hrs.

5. The method for preparing a lyophilized collagen powder according to claim 1, wherein in step (2), further, the temperature during low-temperature crushing is −30° C. to −5° C.; and preferably, the temperature during low-temperature crushing is −10° C.

6. The method for preparing a lyophilized collagen powder according to claim 1, wherein in step (3), during the quick lyophilization, the pre-freezing temperature is −20° C. to −80° C., the pre-freezing time is 4 to 12 hrs, and the lyophilizing time is 48 to 96 hrs; and preferably, the pre-freezing temperature is −30° C.; the pre-freezing time is 6 hrs, and the lyophilizing time is 72 hrs.

7. The method for preparing a lyophilized collagen powder according to claim 1, wherein in step (4), the weight ratio of the solid contents in the collagen solutions in Step (1) and Step (4) is 1:1.5-1:2, and the low-temperature stirring temperature is 2° C.-10° C.

8. The method for preparing a lyophilized collagen powder according to claim 2, wherein freezing irradiation at a low temperature in the presence of dry ice is adopted, where the dose of irradiation sterilization is 10 to 25 KGy, and the temperature is −10° C. to −70° C.
